(19) United States
(12) Patent Application Publication
(10) Pub. No.: US 2024/0186012 A1
(43) Pub. Date: Jun. 6, 2024

(54) SOCIAL DETERMINANT OF HEALTH RISK INDEX FOR STRATIFYING A RISK OF AN ADVERSE HEALTH OUTCOME ACROSS LOCALITIES OF INTEREST

(71) Applicant: Health Solutions Research, Inc., Rockville, MD (US)

(72) Inventors: Ajay Kumar Gupta, Potomac, MD (US); Ramani Peruvemba, McLean, VA (US)

(21) Appl. No.: 18/061,686

(22) Filed: Dec. 5, 2022

(51) Int. Cl. G16H 50/30; G16H 10/60; G16H 40/67

(52) U.S. Cl. CPC G16H 50/30; G16H 10/60; G16H 40/67

(57) ABSTRACT

A method and system for providing health risk analytics that includes obtaining social, economic, and/or environmental data from at least one data source for localities of interest, removing source identification information from the data, determining adverse health outcomes for the localities of interest, identifying an impact of social, economic, and/or environmental factors on the adverse health outcomes based on the set of data, determining which of the social, economic, and/or environmental factors have a greatest impact on the adverse health outcomes for a group of people in the localities of interest to determine a social determinants of health risk index for the localities of interest, as well as determining the degree of impact the social, economic, and/or environmental factors have towards the adverse health outcome. The method also includes determining a health vulnerability score for the adverse health outcomes, and mitigating an effect of the social, economic, and/or environmental factors.

### FIELD OF THE DISCLOSURE

The present disclosure is generally related to systems and methods for providing health risk analytics; in one or more example embodiments, to identify an impact of social, economic, and/or environmental factors on adverse health outcomes, and quantify the degree of impact those social factors have on health outcomes to stratify a risk of the adverse health outcome across a population; and to mitigating an effect of the social, economic, and/or environmental factors having the greatest impact on the adverse health outcomes.

### BACKGROUND

Social determinants of health (SDOH) may be economic and social factors that influence differences in health for a person or group of people. See Social Determinants of Health. Wikipedia [online]. Last edited Sep. 30, 2022 [retrieved on Oct. 7, 2022]. Retrieved from the Internet: <URL: https://en.wikipedia.org/wiki/Social_determinants_of_health> (incorporated herein by reference). For example, the SDOH may include factors such as living and working conditions, e.g., distribution of income, wealth, or neighbourhood, and typically do not include individual risk factors, e.g., behavioural risks/personal choice or genetics/biological data.

It is a growing understanding that individual health outcomes may be more impacted by the SDOH that influences a person's life than the specific healthcare services received and/or lifestyle choices. While a number of indices portend the use of the SDOH, e.g., Social Vulnerability Index, the Area Deprivation Index, or the like, e.g., from the Center for Disease Control and Prevention and/or the University of Wisconsin School of Medicine and Public Health, such indices are intended for a particular interpretation and not reflective of only the impact of the SDOH on the adverse health outcomes and cannot be directly applied to point-of-care decision support with respect to diagnosis, and treatment, nor support public health decision with respect to mitigation, or prevention of the adverse health outcome for localities and/or populations of interest. Moreover, such indices are not granular enough to reflect the local and neighbourhood-by-neighbourhood variations in the SDOH for specific localities of interest and/or population.

That is, while a number of the different indices may include an analysis of the SDOH, there is no system or method presently available that can identify specific SDOH or other factors that have the greatest impact on the adverse health outcomes, especially in which the specific SDOH or other factors having the greatest impact may be easily understandable and able to be displayed with the paired viewability from higher level to granular level understanding of the SDOH, e.g., from a global or national or state level to a neighborhood-by-neighborhood or a Census tract level. In addition, there is no system or method presently available that can identify the degree to which specific SDOH or other factors impact health outcomes in a manner that informs the degree of improvement (e.g., reduction) in the adverse health outcome should the specific SDOH factor be mitigated.

### SUMMARY

The present disclosure is generally related to systems and methods for providing health risk analytics; in one or more example embodiments, to identify an impact of social, economic, and/or environmental factors on adverse health outcomes, and quantify the degree of impact those social factors have on health outcomes to stratify a risk of the adverse health outcome across a population; and to mitigating an effect of the social, economic, and/or environmental factors having the greatest impact on the adverse health outcomes.

In at least one example embodiment, an AI-enabled, geospatial platform is provided that may curate data globally and provide actionable health risk insights throughout the healthcare ecosystem and to health decision makers and policy-makers.

In at least one example embodiment, a method of providing health risk analytics includes obtaining a set of social, economic, and/or environmental data (e.g., data not related to biological data or personal choice data) from at least one data source for localities of interest, removing source identification information from the set of social, economic, and/or environmental data, obtaining adverse health outcomes (e.g., length or quality of life) for the localities of interest, identifying an impact of social, economic, and/or environmental factors on the adverse health outcomes based on the set of social, economic, and/or environmental data, and determining which of the social, economic, and/or environmental factors have a greatest impact on the adverse health outcomes for a group of people in the localities of interest to determine social determinants of health risk index for the localities of interest (e.g., neighborhoods, communities, Census tract level, or the like). The method also includes determining a health vulnerability score for the group of people in the localities of interest for the adverse health outcomes based on the social determinants of health risk index, and displaying an effect of the social, economic, and/or environmental factors having the greatest impact on the adverse health outcomes for the group of people in the localities of interest having highest health vulnerability scores.

In an embodiment, the method may further include removing any geospatial data from the set of social, economic, and/or environmental data.

In another embodiment, the obtaining the set of social, economic, and/or environmental data is through at least one of an application programming interface, webscraping, direct download from a source site, and cloud objects.

In another embodiment, the identifying the impact of social, economic, and/or environmental factors on the adverse health outcomes includes transforming the set of social, economic, and/or environmental data into a risk signal associated with the set of social, economic, and/or environmental data.

In another embodiment, the localities of interest include at least one of a nation, a state, a region, a county, a ZIP code, a Census tract, or a hospital service delivery area.

In an embodiment, the social determinants of health comprise a combination of one or more of the following childhood development, quality of life, education level, housing density, neighbourhood, family income, race, and ethnicity.

In an embodiment, the determining which of the social, economic, and/or environmental factors have the greatest impact includes leveraging a bi-variate cluster analysis to identify the specific social, economic, and/or environmental factors impacting the adverse health outcomes.

In an embodiment, the method further includes reattaching the geospatial data to the health vulnerability score, and providing a mapping and/or visual geospatial representation of the health vulnerability score at different geographical levels for the localities of interest. In an embodiment, the method further includes predicting a triggering event from the social, economic, and/or environmental factors for the adverse health outcomes based on changes of the social, economic, and/or environmental factors.

In yet another embodiment, the obtaining the set of social, economic, and/or environmental data further includes processing image data, which includes processing a single image or by combining a set of images and processing the combined set of images.

In an embodiment, the processing of the image data includes converting the set of social, economic, and/or environmental data into tabular form after removing any of the geospatial data from the image data.

In an embodiment, data from the set of social, economic, and/or environmental data from the at least one data source is geocoded.

In an embodiment, the method further includes displaying the localities of interest having the highest health vulnerability scores and the social determinants of health associated with the highest health vulnerability scores, and when the localities of interest change based on any change to the localities of interest having the highest health vulnerability scores, displaying any updated localities of interest having a new highest health vulnerability score and new social, economic, and/or environmental factors associated with the new highest health vulnerability score.

It will be appreciated that the output (including health vulnerability scores, SDOH, resource levels, etc.) of the predictive model may also be integrated into a data processing system including software systems supporting decision makers, including, but not limited to local or state level government agencies or policy-makers, disaster and emergency response platforms and management systems, electronic health records system, other clinical application, supply chain tracking and management software system, or any other suitable data processing system.

In at least one example embodiment, a non-transitory computer-readable medium has computer-readable instructions that, if executed by a computing device, cause the computing device to perform operations including the above methods and/or other methods disclosed herein.

In another embodiment, a system for providing health risk analytics includes a plurality of data sources having social, economic, and/or environmental data, a cloud-based system configured to obtain a set of the social, economic, and/or environmental data from the plurality of data sources (e.g., including, but not limited to, using a Lambda function to call specific algorithms or functions or SQL search strings). The cloud-based system includes machine-learning algorithms (e.g., machine learning algorithms, artificial intelligence, or other data science techniques built into the SQL searches), which are configured to remove source identification information from the set of social, economic, and/or environmental data, obtain adverse health outcomes from localities of interest, identify an impact of social, economic, and/or environmental factors on the adverse health outcomes based on the set of social, economic, and/or environmental data, determine which of the social, economic, and/or environmental factors have a greatest impact on the adverse health outcomes for a group of people in the localities of interest to determine a social determinants of health risk index for the localities of interest, and determine a health vulnerability score for the group of people in the localities of interest for the adverse health outcomes based on the social determinants of health risk index. The system may also include a relational cloud-based server configured to receive the health vulnerability score for the localities of interest and the associated social, economic, and/or environmental factors having the greatest impact on the adverse health outcomes for the group of people, wherein the relational cloud-based server is configured to transmit the health vulnerability score for the localities of interest and the associated social, economic, and/or environmental factors to application program interfaces for display by an end user. The system may also be configured to allow users to select specific SDOH or other factors and view the effect of the selected SDOH or other factors for the localities of interest. For example, a realtor may select SDOH factors related to education level, income, and quality of life for a specific locality of interest for a neighborhood to view the effects of the specific SDOH.

By leveraging the geospatial tracking capabilities, artificial intelligence based predictive analytics, and social, economic, and/or environmental data sets, the method and system may understand which SDOH or other factors have the greatest impact on the health outcomes and identify at-risk areas having the highest health vulnerability scores. The modelling may follow a phased approach (e.g., local, regional, national, and international or global) that expands or contracts on both the geographic region and the Census tract level and improves accuracy of the prediction in each successive phase. The method and system may allow decision makers including government agencies, policy makers, and health systems and health authorities to target specific SDOH or other factors that have the greatest impact on adverse health outcomes for specific localities of interest and vulnerable groups of people, to mitigate or prevent the impact of the SDOH or other factors on the vulnerable population. It will also be appreciated that the output of the method and system (e.g., health vulnerability score, SDOH or other factors, etc.) may be integrated into software systems.

By accurately assessing which SDOH or other factors have the greatest impact on the most vulnerable of populations, determinations as to which SDOH or other factors provide the greatest barriers of the social and/or economic inequities may be made so that prudent decisions may be made as to provide true and lasting solutions for the diagnosis or treatment of certain adverse health outcomes. Accordingly, the method and system may facilitate upward mobility of our most vulnerable population and remove the social and/or economic inequities in our society.

It will be appreciated that the above embodiments are merely illustrative of the technical concept and features of the method and system for providing health risk analytics, and these embodiments are to provide a person skilled in the art with an understanding of the contents of the method and system for providing health risk analytics in order to implement the method and system for providing health risk analytics without limiting the scope of protection. Any features described in one embodiment may be combined with or incorporated/used into the other embodiment, and vice versa. The equivalent change or modification according to the substance of the method and system for providing health risk analytics should be covered by the scope of protection of the method and system for providing health risk analytics.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

The present disclosure provides a detailed and specific description that refers to the accompanying drawings. The drawings and specific descriptions of the drawings, as well as any specific or alternative embodiments discussed, are intended to be read in conjunction with the entirety of this disclosure. The pandemic and infectious/contagious disease response engine may, however, be embodied in many different forms and should not be construed as being limited to the embodiments set forth herein; rather, these embodiments are provided by way of illustration only and so that this disclosure will be thorough, complete and fully convey understanding to those skilled in the art.

References are made to the accompanying drawings that form a part of this disclosure and which illustrate embodiments in which the systems and methods described in this specification may be practiced.

FIG. 1 is a schematic diagram illustrating the cyclic relationship and effect of health factors and policies and programs on health outcomes, according to at least one example embodiment described herein.

FIG. 2 is a schematic diagram of a health risk analytics system, according to at least one example embodiment described herein.

FIG. 3 is a schematic diagram of a health risk analytics system, according to at least one other example embodiment described herein.

FIG. 4 shows an example table generated by a health risk analytics system and identification of social, economic, and/or environmental factors having the greatest impact on the adverse health outcomes.

FIG. 5 shows an example map generated by a health risk analytics system, according to at least one example embodiment described herein.

FIG. 6 shows another example map generated by a health risk analytics system, according to at least one example embodiment described herein.

FIG. 7 shows another example map generated by a health risk analytics system, according to at least one example embodiment described herein.

FIG. 8 shows another example map generated by a health risk analytics system, according to at least one example embodiment described herein.

### DETAILED DESCRIPTION

The present disclosure is generally related to systems and methods for providing health risk analytics; in one or more example embodiments, to identify an impact of social, economic, and/or environmental factors on adverse health outcomes, and quantify the degree of impact those social factors have on health outcomes to stratify a risk of the adverse health outcome across a population; and to mitigating an effect of the social, economic, and/or environmental factors having the greatest impact on the adverse health outcomes.

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a”, “an” and “the” include plural references unless the context clearly dictates otherwise. Although any systems and methods similar or equivalent to those described herein may be used in the practice or testing of embodiments of the present disclosure, the preferred, systems and methods are now described.

“Public Health Decision Maker” or “Decisions Maker” as discussed herein includes health departments at the Federal, State, County, and City levels; health systems, such as hospitals; health regulators/legislators; front-line clinicians; emergency medical responders; and businesses who manage and adapt to the health concerns of their employees and consumers.

“Vulnerable Populations” as discussed herein refers to those populations vulnerable to poor or adverse health outcomes. Populations can be the overall population as well as defined sub-populations, such as by area including neighborhood, race/ethnicity, culture, insurance coverage, age, gender, or any other attribute of interest.

As discussed herein, the term “health” may be defined as a state of complete physical, mental, and social well-being and not merely the absence of disease or infirmity. Health may influence an individual's ability to reach his or her full potential in society. For example, when the fight-or-flight reaction is chronically elicited in response to constant threats to income, housing, and food availability, the immune system may be weakened, insulin resistance may be increased, and lipid and clotting disorders may appear more frequently. Moreover, mental well-being may influence a person's health. For example, if a person has a perception and experience of one's status in unequal society may lead to stress and poor health. For example, feelings of shame, worthlessness, and envy may lead to harmful effects upon neuro-endocrine, automatic and metabolic, and immune systems.

In at least one example embodiment, the methods and systems discussed herein may provide a generic index that shows or demonstrates the plain impact of the SDOH on the adverse health outcome. In an embodiment, the generic index may be referred to as a SDOH Risk Index, which may assist decision makers to identify where in their state or county or Census tract level, SDOH or other factors that have deleterious effects on health outcomes may be found. In an embodiment, the methods and systems may obtain and analyse a plurality, e.g., at least two, at least twelve, at least twenty, at least twenty-four, of different socioeconomic variables in determining the effects of the SDOH or other factors, and any combinations thereof, on the health outcomes and their relationship to other SDOH or other factors.

It will be appreciated that the “SDOH Risk Index” may refer to the magnitude of the effect of the SDOH or other factors that affect health outcomes both positively and negatively. The scores/indices may be quantified (e.g., 1-5, 1-100, etc., with the higher the magnitude scores/indices indicating higher effect). For example, when the risk score is 3 or more over 5 (if the maximum score is 5) (or 60 or more over 100 (if the maximum score is 100)) for an area, the SDOH or other factors has is a high effect; when the risk score is less than 3 over 5 (or less than 60 over 100) for an area, the SDOH or other factors has is a relatively lower effect. Furthermore, when the SDOH Risk Index is negative, the SDOH or other factors positively affect the health outcomes, whereas, when the SDOH Risk Index is positive, the SDOH or other factors negatively affect the health outcomes.

In at least another example embodiment, the methods and systems leverage a bi-variate cluster analysis to identify the specific SDOH or other factors that impact the adverse health outcomes within specific regions as well as identified specific sub-populations to allow for targeted interventions. While the SDOH Risk Index may provide an index value for each of the SDOH or other factors, the methods and systems may also leverage the determination of a health vulnerability score to provide decision makers a simple and actionable score at a granular level of the relative level of risk to poor or adverse health outcomes determined from the SDOH or other factors.

In a non-limiting embodiment, the method and systems may be provided to identify the impact on health outcomes from social factors (the world we live in) and economic factors (e.g., from data source(s)). Since the impact of social, economic, and environmental factors have been found to affect health outcomes, data on health behaviors including lifestyle choices is omitted, as such choices reflect personal choice and not the impact of social, economic, and environment factors. That is, social, economic, and environmental factors may have an effect on a person's health, contributing to both positive and negative outcomes. For example, both mental and physical health, as well as childhood development and quality of life, may be determined by where you live and the natural environment that surrounds a person. Such factors may also affect lifelong measures including educational attainment and income.

A number of these non-biological factors, e.g., those not including hereditary factors, such as obesity, heart health, etc., and health driven factors/behaviours, such as, exercise, smoking, drug use, diet, sexual activity, etc., are intertwined in the fabric of our society and may be identified as a factor that curtails years off a person's life expectancy. As used herein, these non-biological factors, e.g., not including hereditary factors and health driven factors/behaviours, may be referred to as “social determinants of health” (“SDOH”) or other factors, including, but not limited to environmental factors.

The SDOH may include factors related to, including but not limited to, race and ethnicity, socioeconomic status, education or education level, employment/unemployment, income, family and social support, housing density, segregation, gender, early childhood life, social exclusion, addiction, food access, household characteristics, and community safety, all of which may affect your health.

Unfortunately, it has been found that many of the SDOH factors are socially constructed, which may represent a barrier to intergenerational social economic mobility that may be difficult to break into from the sociological idea of lower status(es). The socially constructed barriers may stem from systemic differences in the way resources are allocated, e.g., determined by a group in leadership roles, lack of inclusivity in public health leadership, historic approach to clinical research, and inequitable application of health policy, e.g., based on race and ethnicity or neighbourhood.

For example, in an embodiment, it has been found that African-American mothers have greater rates of pre-term births than white mothers even when adjusted for income. Studies have also shown that lower socioeconomic status highly correlates with greater amount of toxic matter exposure, e.g., high blood lead levels in children having lower socioeconomic status.

As such, the health risk analytics system and method may be used to identify where problematic SDOH or other factors exist and quantify the magnitude of the problem, such that the relevant SDOH factors may be deconstructed and removed. This may allow the mitigation of the adverse health outcome for the most vulnerable population of our society, in which the most vulnerable population of our society have adverse health outcomes that were based, in part, on social, economic and/or natural/environmental factors that are not within their control. The understanding of the SDOH or other factors, their prevalence, their interaction with each other, and their ultimate effect on health outcomes is of dire importance in order to advance public health in this nation and internationally, especially, for those who are the most vulnerable in our society.

In an embodiment, the SDOH Risk Index may provide an index value for each of the SDOH or other factors, such that the methods and systems may be used to leverage the determination of a health vulnerability score to provide decision makers a simple and actionable score at various viewing levels, e.g., expands or contracts at a national level to a granular level, to view the relative level of risk to poor health outcomes determined from the SDOH or other factors, and which is adjustable to view any changes of the effect of the SDOH or other factors on the health outcomes. In an embodiment, the SDOH Risk Index and/or health vulnerability score may help the public health decision makers to identify at least one locality of interest and/or multiple localities of interest in the United States or outside of the United States where there are social and economic (or environmental) inequities and thus signify a significant barrier to care for millions of people.

Embodiments of the present disclosure will be described more fully hereafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

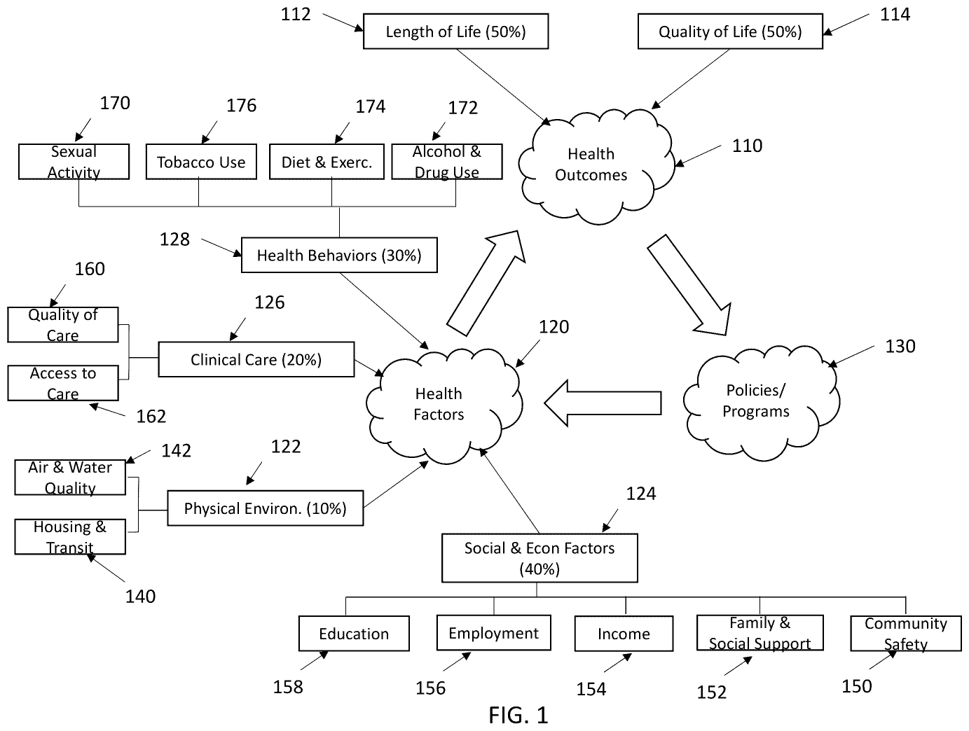

FIG. 1 is a schematic diagram illustrating the cyclic relationship and effect of various health factors and policies and programs on health outcomes. The health outcomes 110 may be positively and negatively affected by a number of various factors including health factors 120, which may be shaped and determined by public policies and programs factors 130. Conversely, the determination of health outcomes 110 may be used to shape policies and/or programs factors 130 for specific localities of interest which may influence or effect various health factors 120 and the overcall health outcome 110 for the localities of interest. The health outcomes 110 may include data of length of life 112, e.g., people living to 75 years or older, and/or from data of quality of life 114, e.g., chronic pains, disabilities or functional limitations. In determining the health outcome, data of the length of life 112 and the quality of life 114 may be equally weighted, e.g., 50%, or unequally weighted, e.g., data related to quality of life weighted more heavily, e.g., 60 to 70%.

FIG. 1 may indicate, based on data obtained about the health factors 120, the magnitude and impact of various health factors that may affect the health outcomes adversely. The health factors 120 may include physical environment factors 122, social and economic factors 124, clinical care factors 126, and health behaviors factors 128. While not illustrated explicitly, it is understood that the schematic diagram of FIG. 1 may be utilized in a processing system connected to databases having the data related to the different factors and a processing system having an AI-enabled platform for determining the effects of the different factors on the health outcomes.

FIG. 1 illustrates that social and economic factors 124 may have a significant impact, e.g., between at least 40% and 60%, on the health outcomes, both positively and negatively, which is greater than the impact of physical environment factors 122, clinical care factors 126, and health behavior factors 128, which may have up to 10%, 20%, and 30% effects on the health outcomes, respectively.

In an embodiment, the physical environment factors 122 may include data of air and water quality 142 and data of housing and transit 140 and may include data of accessibility and/or availability of transportation. In an embodiment, air and water quality 142 may include data that includes average daily density of fine particulate matter in micrograms per cubic meter, per year, radon exposure, and drinking water violations. In an embodiment, transit data may include the percentage of workers who drive alone to work, access to public transit, time and distance of commute or traffic volume.

In an embodiment, social and economic factors 124 may include data of education or education level 158, employment/unemployment 156, income 154, family and social support 152, and community safety 150. The social and economic factors 124 may also include data of housing density, race and ethnicity, segregation, gender, stress, early childhood life, social exclusion, addiction, quality of food and food access, and housing and household characteristics.

In an embodiment, education or education level data may include data of high school completion rates, some high school completion rates, some college attendance rates, number or percentage of college graduates, number or percentage of post-graduate education, preschool enrolment rates, and chronic school absenteeism rates. In an embodiment, the education or education level data may further include percent of population aged 25 years or older with less than 9 years of education, percent of population aged 25 years or older with at least a high school diploma, and percent employed population aged 16 years or older in white-collar occupations.

In an embodiment, employment/unemployment data 156 may include data including percentage of population aged 16 and older unemployed by seeking work, chronic work absenteeism, and job density.

In an embodiment, income data may include median family income in U.S. dollars, income disparity, percent of families below federal poverty level, percent of population below 150% of federal poverty level (e.g., Retrieved from the Internet on Nov. 1, 2022: <URL: https://www.healthcare.gov/glossary/federal-poverty-level-fpl/> or Retrieved from the Internet on Nov. 1, 2022: <URL: https://aspe.hhs.gov/topics/poverty-economic-mobility/poverty-guidelines/prior-hhs-poverty-guidelines-federal-register-references/2021-poverty-guidelines> or Retrieved from the Internet on Nov. 1, 2022: <URL: https://globaldatalab.org/iwi/>), and percent of civilian labour force population aged 16 years and older who are employed.

In an embodiment, family and social support data may include isolation statistics of people, social support statistics from family and friends, children in single-parent households, number of membership associations per 10,000 population, and amount of segregation between different ethnicities.

In an embodiment, community safety data may include number of reported violent crime offenses per 100,000 population, number of deaths due to injury per 100,000 population, number of deaths due to homicide per 100,000 population, number of deaths from self-inflicted injuries per 100,000 population, and number of deaths due to firearms per 100,000 population.

In an embodiment, early childhood life data may include the number of infants having low birth weight or birth weight gap per 10,000, percentage of 3 and 4 year olds in pre-K, vaccination rate, percentage of births to mothers aged 15-19 years, absenteeism, per pupil expenditures, lead paint risk score, percentage of disconnected youth including teens and young adults aged 16-19 who are neither working nor in school, and rate of delinquency cases per 1,000 juveniles.

In an embodiment, housing and household characteristics data may include data of median home value in U.S. dollars, median gross rent in U.S. dollars, median monthly mortgage in U.S. dollars, percent owner-occupied housing units, and percent occupied housing units without complete plumbing. In an embodiment, the housing and household characteristics data may further include percentage of single-parent households with children younger than 18 years, percentage of households without a motor vehicle, percentage of household without a telephone, and percentage of households with more than 1 person per room. In an embodiment, the housing and household data may further include the percentage of household with one or more of the following housing problems: housing quality, housing unit lacks complete kitchen facilities, housing unit lacks complete plumbing facilities, household is overcrowded, household is severely cost burdened, median monthly mortgage which evaluates the number of households that pay more and that pay less than the median mortgage, eviction rate, or access to internet, including access to broadband internet.

In an embodiment, additional social and economic factors 124 may include data that includes recreation and green space availability, and percentage of households with access to healthy foods within 0.5 miles for urban households or 10 miles for rural households, e.g., food deserts, in which health foods includes fresh vegetables or fruits, non-frozen meats, access to non pre-packaged foods, or the like, and percentage of population with food insecurity.

In an embodiment, the clinical care factors 126 may include data of access to care 162 and quality of care 160. The access to care 162 data may include data that includes access to healthcare within a 10-mile radius or ratio of population to healthcare, including pharmacies, primary and secondary care physicians, mental health providers, and dentists. In an embodiment, the quality of care 160 may include data that includes age of physicians or health care providers, satisfaction of physicians and health care providers, and trust of physicians and health care providers. In an embodiment, the radius may be adjusted or specified by the user based on various factors, including, but not limited to, urgency for healthcare as determined by the disease, e.g., heart issues need care more than certain respiratory illnesses.

In an embodiment, health behaviors factors 128 may include data of tobacco use 176, diet and exercise 174, alcohol and drug use 172, and sexual activity 170. While not intending to be limiting, it is understood that the health behaviors factors 128 include data relating to personal choices of the individual.

While the above factors have been discussed, it is understood that the factors are not an exhaustive list of the factors that may be considered. In an embodiment, other factors may be considered that are more important and/or more instructive on the health risks.

The data sources described above may be accessed from publicly and/or privately available data sources, including, but not be limited to a combination of one or more of U.S. Federal Agencies, Non-Profit Organizations, educational institutions, U.S. state and local government agencies, international governments, and international organizations, and Open RX; and other non-profits and government organizations, etc. for obtaining patient data (e.g., age, whether a patient is chronically ill or has underlying health condition, etc.); EHR (electronic health records) systems, social determinants (e.g., for individuals as well as for a community, financial information, education, travel history, habitat, e.g., long-term care facility or private residency). The U.S. Federal Agencies may include, but not limited to, Agency for Toxic Substance and Disease Registry; Centers for Disease Control and Prevention; Centers for Medicare and Medicaid Services; Federal Emergency Management Agency; GeoPlatform; Homeland Infrastructure Foundation-Level Data; National Aeronautics and Space Administration; National Land Cover Database; National Oceanic and Atmospheric Administration; U.S. Agency for International Development; U.S. Bureau of Labor and Statistics; U.S. Census Bureau; U.S. Department of Health & Human Services; U.S. Department of Homeland Security; U.S. Department of Housing & Urban Development; U.S. Department of Transportation; U.S. Forest Service; U.S. Geological Survey; and U.S. National Address Database. The Non-Profit Organizations may include, but not limited to, National Association of Public Safety GIS Foundation; American Cancer Society; American Heart Association; AmeriGEO; Kaiser Family Foundation; Robert Wood Johnson Foundation; The Group on Earth Observations; Humanitarian Data Exchange; Our World in Data; GISAID; Outbreak.info; COVID ACT Now; COVID Tracking Project; and American Hospital Directory. The educational institutions may include, but not limited to, Dartmouth Institute; Stanford University; University of Maryland College Park; Massachusetts Institute of Technology; Johns Hopkins University; and University of California Berkeley. The U.S. state and local government agencies may include, but not limited to, Baltimore City Health Department; Maryland Behavioral Health Administration; US State Departments of Health; Maryland Governor's Office of Crime Control & Prevention; Maryland Vital Statistics Administration; Montgomery County Department of Health & Human Services; Maryland State Board of Elections; Louisiana Department of Health; Florida Department of Health; and New York Department of Health. The international governments may include, but not limited to, Abu Dhabi Department of Urban Planning and Municipalities; Dubai Health Authority; Federal Competitiveness And Statistics Authority, U.A.E.; Ministry of Health, the Kingdom of Saudi Arabia; Oman National Centre for Statistics and Information; China Centers for Disease Control; United Kingdom National Health Service; Republic of Kenya Ministry of Health; Namibia Ministry of Health; Republic of Niger Ministry of Health; Democratic Republic of Congo Ministry of Health; Seychelles Department of Health; Peru Instituto Nacional de Estadística e Informática; and Peru gob.pe Platforma Nacional de Datos Abiertos. The international organizations may include, but not limited to UNICEF; U.N. Office for Disaster Risk Reduction; The World Bank; The World Health Organization; The WHO Africa Region; Global Data Lab; Google; IHME; Doctors Without Borders; Christian Health Services Corps; Global Development Group; Open Street Maps; and USAID. Further, not only are the systems described, recited, and foreseen herein not limited to the data sources listed above, but may also include any number of different data from the plurality of data sources discussed above.

As discussed above and seen in FIG. 1, individual health outcomes may be more impacted by the social and economic factors, e.g., SDOH, that influence a person's life than the specific healthcare services received, e.g., clinical care factors, or lifestyle choices, e.g., health behaviour factors. For example, in an embodiment, both mental and physical health, as well as childhood development and quality of life may largely be determined on where you live and the natural environment that surrounds you. Similarly, these factors play an outsized role in lifelong measures including educational attainment and income. These myriad of non-biological factors are intertwined in the fabric of our society and may reduce a person's life expectancy, e.g., adversely affect health outcomes. As such, understanding the SDOH and other factors, prevalence of the SDOH, interaction between SDOH, and ultimate and cumulative effect of the SDOH and other factors on health outcomes may be important, especially, to advance public health at local, regional, and national or global levels.

For example, in an embodiment, educational factors may adversely affect health outcomes, e.g., life expectancy or quality of life, in which less educated adults report worse general health, a greater number of chronic health conditions, and more functional limitations and disabilities. In an embodiment, a person's neighborhood factors may affect health in which lower income neighborhoods have positive correlations with low birth weight, childhood injury and abuse, and teenage pregnancy risk. In an embodiment, a person's income factors may affect health, in which the higher an income, the greater the life expectancy, e.g., the difference in life expectancy between the top 1% and the bottom 1% of the economic spectrum being 14.6 years. In an embodiment, race and ethnicity factors may affect health, in which African-American mothers suffering greater rates of pre-term births than white mothers even when adjusted for income.

As such, in the methods and systems as described herein, the social and economic factors 124 may be identified and analyzed to determine which SDOH or other factors, e.g., environmental factors, both positively and negatively, contribute to the effect on health outcomes for specific localities of interest, since different localities of interest may have different SDOH or other factors that affect the respective health outcomes. While not intended to be limited in scope, the methods and systems may also include physical environment factors 122 and/or clinical care factors 126 in determining the effect of such factors on health outcomes. Such determination, however, does not include health behaviors factors 128, at least because the health behaviors factors 128 are personal choices or lifestyle choices, as opposed to factors that may not be controllable.

Of the SDOH or other factors that may be identified and analysed include, but not limited to, a combination of one or more of the following: Total Population; Gender; Race, including but not limited to Caucasian, Black, American Indian, Asian, Native Hawaiian or other Pacific Islander, Other Race, Two or more Races, and Hispanic; Age, including, but not limited to, Over 5 years old, Under the age of 10, Between the ages of 10 and 19, Over the age of 16, Between the ages of 20 and 29, Between the ages of 30 and 39, Between the ages of 40 and 49, Between the ages of 50 and 59, Between the ages of 60 and 69, Between the ages of 70 and 79, Over the age of 80, Over the age of 65, and Over the age of 25; Education, including but not limited to, Less than 9th grade, No High School Diploma, High School Diploma, Some College no Degree, Associates Degree, Bachelor's Degree, and Graduate Degree; Military status, including, but not limited to, Civilian Over the age of 18, Military, and Veteran; Employment type, including, but not limited to, Agriculture, forestry, fishing and hunting, and mining, Construction, Manufacturing, Wholesale Trade, Retail Trade, Transportation and warehousing, and utilities, Information, Finance and insurance, and real estate and rental and leasing, Professional, scientific, and management, and administrative and waste management services, Educational services, and health care and social assistance, Arts, entertainment, and recreation, and accommodation and food services, Other services, except public administration, Public Administration; Income levels, including, but not limited to, Income Under 25 k, Income Between 25 and 49 k, Income Between 50 and 74 k, Income Between 75 and 99 k, Income Between 100 and 149 k, Income Between 150 and 199 k, Income Greater than 200 k; and other SDOH and factors that may include Non-Institutionalized Population, Disabled, Not Fluent in English, In Labor Force, Employed, Unemployed, Not In Labor Force, Commuted by driving alone, Commuted by public transport, Total Households, Median Household Income, Households With Earnings, Households With Social Security, Households With Retirement Income, Households With Supplemental Security Income, Households With Cash Public Assistance Income, Households With Food Stamps or SNAP Benefits, Have Health Insurance, Have Private Health Insurance, Have Public Health Insurance, Do Not Have Health Insurance, Occupied Housing Units, Owner Occupied Housing, Renter Occupied Housing, No Home Heating Fuel, Lack of Completed Plumbing, Lack of Completed Kitchen, Low Occupant Density, Normal Occupant Density, High Occupant Density, Mortgage greater than 30 Percent of Income, Rent greater than 30 Percent of Income, Housing Units, Housing Units with potential lead paint, Households with no vehicle, Median Housing Value, Median Rent Costs, Households with Computer Access, Households with Smartphone Access, Households with Broadband Access, Population age 3 to 5, Enrolled in preschool, Single Parent Household, Voting Status, US Citizen by Birth, US Citizen by Citizenship, Foreign Born, Total Land Area, Total Water Area, and Total Area.

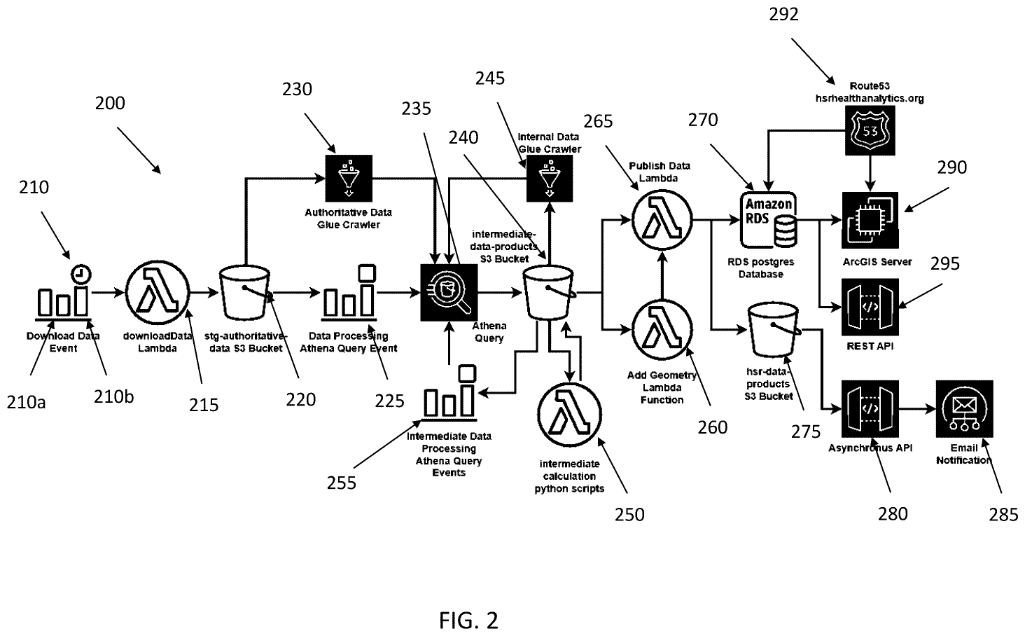

FIG. 2 is a schematic diagram of a health risk analytics system 200, according to at least one example embodiment described herein. The system 200 may include data producers 210, a data pipeline that includes the data processing and analytics, e.g., 215, 235, 240, 245, 250, 255, and data storage, e.g., 220, 270, 275, and data consumers, e.g., 290, 295, 280, 285, for accessing the resulting determinations. The system 200 may be configured to determine which social determinants of health (SDOH), e.g., social and economic factors, or other factors, e.g., environmental factors, affect health outcomes, in which the resulting determination may be adjustable, be provided with expanded or contracted viewing, for viewing at national, global, or regional levels to viewing at various localities or at the census tract level. The system 200 may be a serverless, e.g., cloud-based, query service system, a cloud-based health analysis framework system, a data lake infrastructure that allows for the curation, cleaning, analysis, and dissemination of health risk analytics, a cloud-based spatial data infrastructure, or a cloud-based system with API-enabled accessibility, or a combination of thereof. The system may leverage a bi-variate cluster analysis that includes AI-based learning models to identify the specific SDOH or other factors that impact health outcomes.

FIG. 2 shows a plurality of data sources 210a, 210b, . . . 210n (collectively, “data sources 210” hereafter), which may be communicatively coupled to the health risk analytics system 200. Data sources 210a, 210b, . . . 210n may refer to, but not be limited to, e.g., non-biological data, for example, social, economic, and/or environmental data from a combination of one or more of U.S. Federal Agencies, Non-Profit Organizations, educational institutions, U.S. state and local government agencies, international governments, and international organizations, and Open RX, etc. Further, not only are the systems described, recited, and foreseen herein not limited to the data sources listed above, but they are not limited in quantity to those shown in FIG. 2. Further still, unless context otherwise requires, the description and recitation henceforth may refer to the singular “data source 210” without being limiting.

Data sources 210 may provide, for example, information of health outcomes including length of life and quality of life at the global, regional or state level, and for the local community, including at the neighborhood-by-neighborhood or census tract level or satellite imagery of particular localities of interest or images from ground level. Data sources 210 may also include social, economic, and/or environmental factor data present in specific localities of interest, e.g., nation, state, region, county, ZIP code, Census tract, hospital service delivery areas, etc. For example, in an embodiment, the data is relating to one or more of geography, demographics, local transportation, finances (both macro- and micro-), health care availability, law enforcement resources, social media of individuals, socioeconomic statistics, and/or historical health data. As such, the data sources provide information that is not related to personal choice factors, e.g., behaviors and lifestyles, but rather, the social, economic, and environment of the locality of interest.

The various data sources 210 may be accessed and obtained by the data pipeline of the health risk analytics system 200 by querying the data sources 210, for example, but using a fetcher function having a Lambda function 215, e.g., Python script, having for example, a SQL search string to obtain a set of the social, economic, and/or environmental data for the localities of interest, e.g., regional, state, neighborhood-by-neighborhood, or Census tract level, or a combination thereof. For example, the Lambda function 215 may be configured to call a function and/or algorithm to direct the obtainment of data from any number of the available data sources around the world by focusing on the localities of interest that are more at risk than others, e.g., where risk is high. In another embodiment, the data may be selectively obtained over the worldwide web and accessed using APIs, web scraping functionalities, direct download of files, and other cloud-based objects. The data may be stored in an authoritative bucket 220, as raw data, e.g., data from the data sources without any further processing.

The data pipeline of the health risk analytics system 200 may further include a data processing query event 225 that is configured to scrub data, e.g., metadata, from the data in the authoritative bucket 220. As such, the data from the data sources 210 does not include metadata, e.g., source identifying data, such that the data that is processed in the health risk analytics system 200 is secured and the data source may remain anonymous, e.g., source identifying information is removed. In an embodiment, the data processing query event 225 may include an algorithm that is configured to convert the data into a risk signal, in which the underlying data is erased for further security and anonymity of the data and source data. For example, the data processing query event 225 may invoke a signal extraction function, e.g., trending of the data in a positive or negative direction, such that the risk of the factor may still be assessed, but the context of the data is removed, e.g., to provide security of the data, for example, child abuse data.

In an embodiment, a crawler function 230 may be used that is configured to obtain the data from the authoritative bucket 220 to populate the data for analysis in the health risk analytics system 200. The crawler function 230 may be configured to convert the data into tabular form in which any geospatial data is removed. The crawler function 230 may be configured to crawl the data sources 210 in a single run such that the crawler function 230 may determine the format, schema, and associated properties in the raw data in the authoritative bucket 220, group the data into tables or partitions, and write or add data for analysis. It is appreciated that the geospatial data may include data about objects, events, or phenomena that have a location on the surface of the earth, e.g., a geographic component, that may include geometric data, e.g., data as 2D and/or 3D vectors as points, lines, and polygons in a space, cartographic representations, and/or other positional relationship data.

The data processing query event 225 and/or the crawler function 230 may then be configured to populate the respective transformed data to a Query function 235 having memory to store the data, for example, in a hive data format, e.g., suitable format for processing in intermediate data bucket 240.

In a non-limiting example, the Query function 235 may be connected in a loop with the intermediate data bucket 240, an internal crawler function 245, an intermediate calculation function 250, and an intermediate data processing event function 255, in which a number of different processing steps may occur on the intermediate data. The Query loop may include a plurality of artificial intelligence based Machine-Learning Models that are included in search functions, e.g., SQL search strings, that is configured to access the hive data and perform at least the following functions/processing. In an embodiment, the Query loop may be configured to remove erroneous data, ensure the data has proper geocoding data and format, make necessary corrections to an imagery data obtained, e.g., atmospheric data correction of satellite imagery, for example, remove cloud cover, process any imagery data, e.g., using ENVI or ARTS software, stitch any imagery data together, label and identify the data, e.g., tagged data from the web or identify the data, e.g., as a wildfire. In an embodiment, the Query loop may be configured to convert the imagery data and/or combine imagery data together and covert the image data into tabular form, e.g., using ENVI or ARTS software. As such, the image data may be processed using the Query function 235 to determine the effect of the SDOH or other factors presented in the image data on the adverse health outcomes. For example, in an embodiment, the image data may include images of deforestation or gentrification of a locality of interest. As such, such data may be converted into tabular form and then analysed using the AI-enabled ML algorithm to determine the effects thereof.

The Query function 235 may include query search strings having ML-based models that are configured to perform analysis at the global level, e.g., on the hive data, in the intermediate data bucket 240. In an embodiment, the query search string may be a SQL search that may be used to identify the social, economic, and/or environmental factors having the greatest impact on the health outcomes at a global or high level of the data in the intermediate data bucket 240.

In an embodiment, the internal crawler function 245 may be configured to obtain the data from the intermediate data bucket 240 and may be configured to convert the data into a form for data analysis and back into tabular form. The crawler function 245 may be configured to determine the format, schema, and associated properties in the intermediate data in the intermediate data bucket 240, group the data into tables or partitions, and write or add data for analysis.

The intermediate calculation function 250 may include a plurality of functions/algorithms, including, but not limited to, a ML/AI algorithm or other data science technique for bi-variate cluster analysis that are configured to identify the social, economic, and/or environmental factors that have the greatest impact on the health outcomes for the particular locality of interest. The bi-variate cluster analysis may be configured to identify features or factors in the data that are as similar as possible, and/or that are different as possible, e.g., effect on health outcomes at various levels of the localities of interest, e.g., global, national, stage, or regional and ZIP Code, neighborhood-by-neighborhood or Census tract level. The bi-variate cluster analysis may also include a varying coefficient model, weighted distance measuring model, K-means and hierarchical clustering models, or the like for processing the data. For example, a series of the ML/AI algorithms having the bivariate cluster analysis may be run, for example, an inference function in which the endpoint, e.g., adverse health outcomes, is identified and the social, economic, and/or environment factors in the data are identified to pass the model, e.g., using Amazon SageMaker to deploy various functions and/or ML/AI algorithms. The ML/AI algorithm may also be configured to determine which of the social, economic, and/or environmental factors have the greatest impact on the health outcome, and specifically, for a group of people in the localities of interest, for example, using a bi-variate cluster analysis function. In an embodiment, the data is gathered and analysed for the different levels of the localities of interest such that the data is available or accessible for the different levels of the localities of interest.

In an embodiment, the intermediate calculation function 250 may also include a ML algorithm that is configured to determine a social determinants of health risk index (SDOH Risk Index) for the localities of interest which identifies the social, economic, and/or environmental factors having the greatest impact on the health outcomes. For example, in an embodiment, the intermediate calculation function 250 may identify the SDOH or other factors that positively or negatively influence the health outcome, in which the risk index for each of the SDOH or other factors are determined using a the bi-variate cluster analysis or iterative correlation and regression analysis functions. In an embodiment, the ML algorithm may be an AI-enabled ML algorithm which is configured to iteratively learn from prior SDOH Risk Index determinations on which factors have the greatest impact, both positively and negatively, on the health outcomes.

In an embodiment, the intermediate calculation function 250 may further include a ML/AI algorithm to determine a health vulnerability score for the group of people in the localities of interest based on the SDOH Risk Index scores having the highest values. The health vulnerability score may be determined by weighting these factors based on their overall impact on the health outcome. In an embodiment, the weighting is based on literature review of academic, even weighting, weights assigned based on experiences of clinicians or public health decision maker's experience, other mechanisms, and public health research. The weights may then be adjusted and/or redefined by the iterative correlation and/or regression analysis to using the ML algorithms to determine the effect of the SDOH or other factors on the overall impact on the health outcome. The weights may then be used to calculate the health vulnerability score of the population, on a per-locality basis, e.g., the health vulnerability score for this ZIP Code and for its neighboring ZIP Code. In an embodiment, the users may also be able to select any set of one or more SDOH or other factors to see how those factors impact health outcomes and the amount of contribution to health vulnerability they pose.

In an embodiment, in view of the size of the data available from the various data sources 210, geospatial data may be removed from the data using at least one of the Query function 235, e.g., via SQL string, and the intermediate data processing event function 255. For example, since during the data processing the geospatial data may be added as a column to the data tables, by removing the geospatial data from the data, the processing efficiency of the health risk analytics system 200 may be improved, at least because the geospatial data would not have be included in any of the calculations or ML/AI-based model processing algorithms. In an embodiment, the geospatial data may include spatial data for mapping on a two-dimensional or three-dimensional surface, for example, including, but not limited to, property data including distance, shape, size, relative position, topology of features and boundaries related to mapping. The geospatial data may also include data relating to the specific localit(ies) of interest.

In an embodiment, the intermediate calculation function 255 may also be configured to remove erroneous data, ensure the data has proper geocoding data and format, make necessary corrections to an imagery data obtained, e.g., atmospheric data correction of satellite imagery, for example, remove cloud cover, process any imagery data, e.g., using ENVI or ARTS software, stitch any imagery data together, label and identify the data, e.g., tagged data from the web or identify the data, e.g., as a wildfire.

In an embodiment, the intermediate data bucket 240 may be accessed by an add geometry function 260 which is configured to convert the data in tabular form from the intermediate data bucket 240. The add geometry function 260 may also be configured to add any geospatial data that was previously removed by the Query function 235 and/or the intermediate data processing event function 255. A publish data function 265 may be used to publish the data processed by the query loop, e.g., the identification of the SDOH or other factors that have the greatest impact on health outcomes and their relative impact thereof, the SDOH Risk Index for each SDOH or other factors, the health vulnerability score, and associated localities of interest at various viewing levels. The publish data function 265 may be configured to send or receive a signal to send the SDOH or other factors, the SDOH Risk Index, the health vulnerability score, etc. to a relational database service, e.g., RDS web-accessible database 270, and/or a data products web-accessible server 275 that includes databases in the cloud such that the data may be accessed by an end user and/or a public health decision maker for displaying on a display. In an embodiment, the data may be accessed by an asynchronous API 280 that is connected to an email server 285 to send the data or link to the data to the end user or the public health decision maker. In an embodiment, the RDS web-accessible database 270 may be accessible via a server 290 for providing the data to the end user and/or public health decision maker via a web-accessible server or a REST API 295. In an embodiment, the RDS web-accessible database 270 may be connected to the server 290 via a host server 292. As such, the host server 292 may direct traffic or provide accessing credentials for allowing access to the RDS web-accessible server 270, e.g., username, password, biometrics, encrypted access, or the like.

In an embodiment, the SDOH or other factors and their relative impact thereof, the SDOH Risk Index, the health vulnerability score, etc., are available for download or accessible through at least one of Esri Marketplace as a Geospatial Layer (or Feature Service), SDI through open standards, including but not limited to WMS, WFS, OGC API, etc., as a CSV or through RESTful and Asynchronous API access, the Esri ArcGIS Server or other geospatial application, or may be viewed as an ArcGIS StoryMap, or an ArcGIS Insights Dashboard, or can be embedded on a public or private (e.g., password-protected) website or web portal. Such access to the data allows the health risk analytics system 200 to be configured to adjust the viewability of the SDOH or other factors, the SDOH Risk Index, the health vulnerability score, etc., e.g., from higher levels to granular levels, e.g., from global, national or state level to neighborhood-to-neighborhood or Census tract level, to easily and visually understood the impact on the health outcomes so that the decision makers may make actionable decisions to mitigate an effect of the social, economic, and/or environmental factors on the group of people having the highest health vulnerability scores, e.g., quantify the degree of impact of those SDOH or other factors on health outcomes. That is, by identifying the impact of the SDOH or other factors on the adverse health outcomes, a value, e.g., dollar amount, may be quantified and the return, e.g., economic value, may be determined to assist decision makers in quantifying the effect of the SDOH or other factors for improvement thereof.

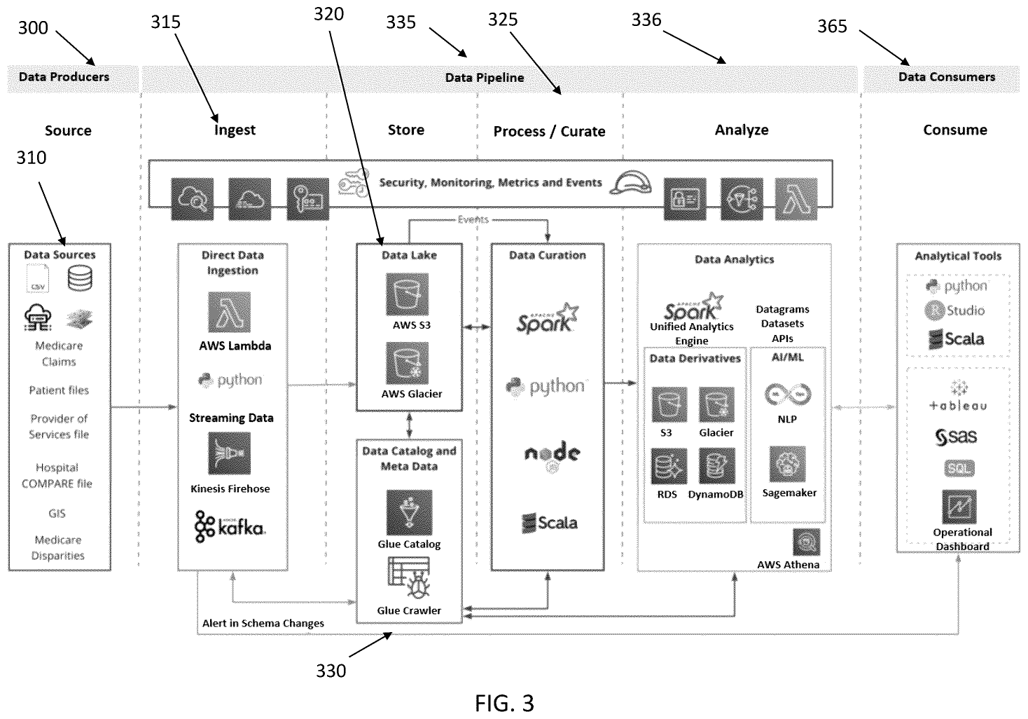

FIG. 3 illustrates a non-limiting example of a health risk analytics system 300 configured to identify the impact of SDOH and other factors on health outcomes, e.g., from social, economic, and/or environmental factors from the world we live in. The health risk analytics system 300 may include data producers 310, e.g., a plurality of database(s), data pipeline which may include a processing system 335 for analyzing and transmitting the data, and data consumers 365, e.g., end-users using API or web-accessible servers to make decisions related to the health outcomes, e.g., public health decision makers.

The processing system 335 that may be used to obtain a set of social, economic, and/or environmental data from at least one data source 310 for a particular locality of interest. The data sources 310 may be the same or similar data sources 210a, . . . 210n of FIG. 2. The data received from one or more instances of data sources may include, but not be limited to vital health statistics and social determinants of health resources that are available on municipality-levels, e.g., global, national level, state level, counties, cities, towns, ZIP codes, Census tracts, and Census blocks, or hospital service delivery area. The data is provided by data sources 310 may also include data that includes various socio-economic and environmental and non-biological factors for those in position to provide assistance, such as, e.g., public health decision makers. Those factors may include, but not be limited to: environmental and/or climate change related data, population demographics, education, and/or racial disparities that may contribute to access to quality health services; socio-economic variables such as social class, income, access to food, housing, etc.

The various data sources 310 may be accessed and obtained by the processing system 335 by querying the data sources 310, for example, by including and using an ingestion/fetcher function 315. The ingestion function 315 may include a Lambda function, e.g., Python script, having a SQL search string to access and obtain a set of social, economic, and/or environmental data for the localities of interest from the data sources 310. In an embodiment, the ingestion function 315 may additionally include or include an extract, transform, and load service, e.g., Apache Kafka, Amazon Kinesis Firehouse, or the like, to obtain and access the data sources 310 as streaming data. The data may be stored in an authoritative bucket 320, e.g., data lakes for storage on cloud-based servers.

The processing system 335 may further include a data processing and curation function 325 that may be configured to scrub data, e.g., metadata, from the data in the authoritative bucket 320. As such, the data from the data sources 310 does not include metadata, e.g., source identifying data, such that the data that is processed in the processing system 335 is secured and the data source may remain anonymous, e.g., source identifying information is removed. In an embodiment, the data processing and curation function 325 may be configured to convert the data into a risk signal, in which the underlying data is erased. For example, the data processing and curation function 325 may invoke an algorithm that is configured as a signal extraction function, e.g., trending of the data in a positive or negative direction, such that the risk of the factor may still be assessed, but the context of the data is removed, e.g., to provide security of the data. The data processing and curation function 325 may include algorithms written in object-oriented programming and functional programming scripts, e.g., Scala, Python, Node (Java), and/or accessed by open-source, distributed processing system, such as, Apache Spark, etc., for development of APIs in Java, Python, Scala, R, or the like.

In an embodiment, a crawler function 330 may be used that is configured to obtain the data from the authoritative bucket 320 to populate the data for analysis in the processing system 335. The crawler function 330 may be configured to convert the data into tabular form in which any geospatial data may be removed. The crawler function 330 may be configured to crawl the data sources 310 in a single run such that the crawler function 330 may determine the format, schema, and associated properties in the raw data in the authoritative bucket 320, group the data into tables or partitions, and write or add data for analysis.

The data processing and curation function 325 and/or the crawler function 330 may then be configured to populate the respective transformed data for analysis by an analyzing function 336 having memory to store the data, for example, in a hive data format.

The analysing function 336 may be used to generate a SDOH Risk Index which may be an indication of which SDOH or other factors have the greatest impact on adverse health outcomes.

In a non-limiting example, the analyzing function 336 may be a loop that includes a plurality of artificial intelligence based Machine-Learning Models that are included in search functions, e.g., SQL search strings and/or Lambda functions for calling the ML/AI models, that is configured to access the hive data and perform at least the following functions/processing. In an embodiment, the analyzing function 336 may be configured to remove erroneous data, ensure the data has proper geocoding data and format, make necessary corrections to an imagery data obtained, e.g., atmospheric data correction of satellite imagery, for example, remove cloud cover, process any imagery data, e.g., using ENVI or ARTS software, stitch any imagery data together, label and identify the data, e.g., tagged data from the web or identify the data, e.g., as a wildfire.

The analyzing function 336 may also include query search strings having ML-based models that are configured to perform at least a bi-variate cluster analysis to identify the social, economic, and/or environmental factors having the greatest impact on the health outcomes. In an embodiment, the bi-variate cluster analysis may be configured to identify features or factors in the data that are as similar as possible, and/or that are different as possible, e.g., effect on health outcomes at various levels of the localities of interest, e.g., global, national, state, or regional and ZIP Code, neighborhood-by-neighborhood or Census tract level. The bi-variate cluster analysis may also include a varying coefficient model, weighted distance measuring model, K-means and hierarchical clustering models, or the like for processing the data.

The analyzing function 336 may include, in an embodiment, the loop having at least one of an internal crawler function, an intermediate calculation function, and an intermediate data processing event function. In an embodiment, the analysing function 336 may be connected to the data processing and curation function 325 and/or the crawler function 330.

The analyzing function 336 may include a ML/AI or data science algorithm that is configured to identify the social, economic, and/or environmental factors that affect the health outcomes for the particular locality of interest. For example, the ML/AI algorithm may run an inference function in which the endpoint, e.g., adverse health outcomes, is identified and the social, economic, and/or environment factors in the data are identified to pass the model, e.g., using Amazon SageMaker to deploy the various algorithms/functions. The ML/AI algorithm may also include the SQL search strings in which the ML algorithm is configured to determine which of the social, economic, and/or environmental factors have the greatest impact on the health outcome, and specifically, for a group of people in the localities of interest, for example, using a bi-variate cluster analysis function. In an embodiment, the data is gathered and analysed for the different levels of the localities of interest such that the data is available or accessible for the different levels of the localities of interest.

In an embodiment, the analyzing function 336 may also include a ML/AI algorithm that is configured to determine a social determinants of health risk index (SDOH Risk Index) for the localities of interest which identifies the social, economic, and/or environmental factors having the greatest impact on the health outcomes. For example, in an embodiment, the analyzing function 336 may identify the SDOH or other factors that positively or negatively influence the health outcome, in which the risk index for each of the SDOH or other factors are determined using a the bi-variate cluster analysis or iterative correlation and regression analysis functions. In an embodiment, the ML algorithm may be an AI-enabled ML algorithm which is configured to iteratively learn from prior SDOH Risk Index determinations on which factors have the greatest impact, both positively and negatively, on the health outcomes.

In an embodiment, the analyzing function 336 may further include a ML/AI algorithm to determine a health vulnerability score for the group of people in the localities of interest based on the SDOH Risk Index scores having the highest values. The health vulnerability score may be determined by weighting these factors based on their overall impact on the health outcome. In an embodiment, the weighting is based on literature review of academic, even weighting, weights assigned based on experiences of clinicians or public health decision maker's experience, other mechanisms, and public health research. The weights may then be adjusted and/or redefined by the iterative correlation and/or regression analysis to using the ML algorithms to determine the effect of the SDOH or other factors on the overall impact on the health outcome. The weights may then be used to calculate the health vulnerability score of the population, on a per-locality basis, e.g., the health vulnerability score for this ZIP Code and for its neighboring ZIP Code. In an embodiment, each SDOH or other factor may be weighted to reflect the degree of its contribution to the SDOH Risk Index, in which the weight may be a representation of the level of the SDOH or other factor's contribution to the overall impact (risk) to health outcomes. In an embodiment, weights can be set to 0 to assess the relative value of different SDOH factors on the overall SDOH Risk Index. In an embodiment, the users may also be able to select any set of one or more SDOH or other factors to see how those factors impact health outcomes and the amount of contribution to health vulnerability they pose.

It is appreciated that the use of a SQL search string with a ML/AI model and/or the use of the Lambda function to call a specific ML/AI model by the analysing function 336 may depend on a number of various factors. For example, if the data to be analysed is the data from the entire intermediate data bucket, the Query function may use a SQL string for analysing the data, since SQL strings are more computationally efficient, and may be used for simpler calculations, e.g., initial processing. On the other hand, since the ML/AI model called by the Lambda function may be used for the bivariate cluster analysis, the data analysed by this ML/AI model may include more specific localities of interest to decrease the data being analysed. As such, the Lambda function may call a plurality of ML/AI models for the bivariate cluster analysis to analyse selected groups of data, e.g., based on localities of interest, to increase computational time and not overburden the computational resources of the cloud-based system.

In an embodiment, in view of the size of the data available from the various data sources 310, geospatial data may be removed from the data by the analyzing function 336 and/or during the data processing and curation function 325. For example, since during the data processing the geospatial data may be added as a column to the data tables, by removing the geospatial data from the data, the processing efficiency of the health risk analytics system 300 may be improved, at least because the geospatial data would not be included in any of the calculations or ML-based model processing algorithms, e.g., would not slow down processing. In an embodiment, the geospatial data may include spatial data for mapping on a two-dimensional or three-dimensional surface, for example, including, but not limited to, property data including distance, shape, size, relative position, topology of features and boundaries related to mapping. The geospatial data may also include data relating to the specific localit(ies) of interest.

In an embodiment, the resulting analytics data from the analysing function 336 may be published to a relational database service, e.g., a RDS web-accessible database, and/or a data products web-accessible server, for access by the data consumers 365 for displaying the SDOH or other factors that have the greatest impact on health outcomes, the SDOH Risk Index for each SDOH or other factors, the health vulnerability score, and associated localities of interest at various viewing levels for displaying on a display, e.g., monitor or display screen. In an embodiment, the data may be accessed by an asynchronous API and/or operational dashboard, e.g., SAS, Tableau, SQL, Amazon Quicksight or the like. In an embodiment, the RDS web-accessible database may be accessible via a server for providing the data to the end user and/or public health decision maker via a web-accessible server or a REST API. In an embodiment, the RDS web-accessible database may be connected to the server via a host server. As such, the host server may direct traffic or provide accessing credentials for allowing access to the RDS web-accessible server, e.g., username, password, biometrics, encrypted access, or the like.

In an embodiment, the SDOH or other factors and their relative impact thereof, the SDOH Risk Index, the health vulnerability score, etc., are available for download or accessible through at least one of Esri Marketplace as a Geospatial Layer (or Feature Service), SDI through open standards, including but not limited to WMS, WFS, OGC API, etc., as a CSV or through RESTful and Asynchronous API access, the Esri ArcGIS Server or other geospatial application, or may be viewed as an ArcGIS StoryMap, or an ArcGIS Insights Dashboard, or can be embedded on a public or private (e.g., password-protected) website or web portal. Such access to the data allows the health risk analytics system 300 to be configured to adjust the viewability of the SDOH or other factors, the SDOH Risk Index, the health vulnerability score, etc., from higher levels to granular levels, e.g., from global, national or state level to neighborhood-to-neighborhood or Census tract level, to easily understood the impact on the health outcomes so that the decision makers may make actionable decisions to mitigate an effect of the social, economic, and/or environmental factors on the group of people having the highest health vulnerability scores, e.g., quantify the degree of impact of those SDOH or other factors on health outcomes. That is, by identifying the impact of the SDOH or other factors on the adverse health outcomes, a value, e.g., dollar amount, may be quantified and the return, e.g., economic value, may be determined to assist decision makers in quantifying the effect of the SDOH or other factors for improvement thereof.

As such, the health risk analytics system 300 may be configured to identify an impact of the SDOH or other factors on the adverse health outcomes. The health risk analytics system 300 may also be configured to determine which of the SDOH or other factors have the greatest impact on the adverse health outcomes for a group of people in the localities of interest.

For example, in an embodiment, the SDOH Risk Index may incorporate data of housing, income, race, education, transportation, health access, voting patterns, and other factors. This index may be calculated at higher levels, from regional or country levels down to the granular level, e.g., Census tract level. The end user may then interact with the health vulnerability score, via a GUI or interface that is displayed on a display, e.g., through a dashboard, where the end user may pick any set of Census tracts to look at to identify which areas have a higher or lower health vulnerability risk value than others and view at various levels associated with the most granular level, e.g., the Census tract level, to compare the effects of different SDOH or other factors at the various levels on the vulnerable population and/or localities of interest. The end users may also select a set of one or more SDOH or other factors to see how those factors impact health outcomes and the amount of contribution to the health vulnerability score they pose. As such, the end user or public health decision maker may be able to mitigate the effect of the SDOH or other factors having the greatest impact on the adverse health outcomes for the group of people in the localities of interest having the highest health vulnerability scores that is quantifiable and justifiable on a monetary basis. For example, in an embodiment, the end user or public health decision maker may allocate additional funds and/or divert resources when the SDOH or other factors related to poor housing and poor education are found to have a disproportioned effect on health outcomes, especially, when such allocations of funds may be quantified with a cost savings and/or economic growth.

In another non-limiting embodiment, since data from the data sources 310 may be obtained and accessed as streaming data, the analyzing function 336 may be configured to perform additional processing based on the real-time data of the streaming data. For example, in an embodiment, the health risk analytics system and method is configured to track the impact of the interventions or mitigation effects in the short- and long-term. This allows for immediate feedback to course correct interventions, helping ensure successful mitigation of the identified health problem. Such tracking may be based on ML models that are configured to predict the effect of the mitigation of the one or more SDOH or other factors at the locality of interest based on prior effects on the health outcome. Such predictions by the ML models of the analyzing function 336 may be used to provide a triggering event determination from the social, economic, and/or environmental factors that either changes the view of the health vulnerability score, e.g., expanded or contracted viewing, to provide the decision maker with an actionable view and value in an attempt to further mitigate the adverse health outcome. The triggering event may be based on threshold values based on the distribution of the risk signal. For example, in an embodiment, the Health Risk Index values below 2.5 standard deviations may be categorized as low risk, whereas, the values above 2.5 standard deviations may be categorized as high risk, which would result in a triggering event. As such, in an embodiment, if SDOH Risk Indexes of certain SDOH or other factors are trending in a positive direction, e.g., negatively affecting health outcomes, the analyzing function 336 may be configured predict the health vulnerability score to trigger an appropriate action by the decision maker.

In order to improve computational efficiency, the processing system 335 may include a number of different functionalities. For example, in an embodiment, when identifying the impact of the social, economic, and/or environmental factors on the adverse health outcomes, the crawler function may be configured to convert the social, economic, and/or environmental data to only a risk signal, e.g., the data itself is not sent for processing, but converted to a risk signal such that the context of the data is not communicated or stored. Since only a risk signal is sent for processing, the processing system 335 may have improved functionalities of processing and data storage. In another exemplary embodiment, the processing system 335 may remove any geospatial data from the social, economic, and environmental data. This may include removing the geospatial data from the tabular data created by the crawler function. Such removal of geospatial data may also improve the computational efficiency of the processing system 335 since such data does not have to be processed by the processing system 335 to determine the SDOH Risk Index or health vulnerability score.

In an embodiment, the processing system 335 may be configured to reattach the geospatial data to the health vulnerability score and provide a mapping and/or visual geospatial representation of the health vulnerability score at different geographical levels for the localities of interest. For example, in an embodiment, the health vulnerability score may be shown as a thematic map showing the risk by area (locality of interest), and can also be presented in tabular and graphical form. The health vulnerability score may be made relative and normalized to fall into any identified range, in which relative implies that a health vulnerability risk score of “Low” or “12” for a particular area is only meaningful when compared to the risk score of another area that may be “medium” or “17”.

In an embodiment, the health vulnerability score may be merged with shape files at multiple geospatial levels (including but not limited to: nation, state, region, county, ZIP code, census tract, hospital service delivery area, etc.) for display. The health vulnerability score may also merged with a symbology to illustrate graduation between High risk, Medium risk, and Low risk, in which the symbology can support a wide range of risk levels, typically but not limited to between 3 and 7 levels.

The example tables and maps generated by the health risk analytics system disclosed herein may show a prediction of which SDOH or other factors may adversely affect the health outcome for particular localities of interest and serve as a tool for assessing the allocation of resources for localities of varying degrees of risk, and for illustrating zones in accordance with at least some embodiments described herein to mitigate the effect of the SDOH or other factors on the adverse health outcomes.

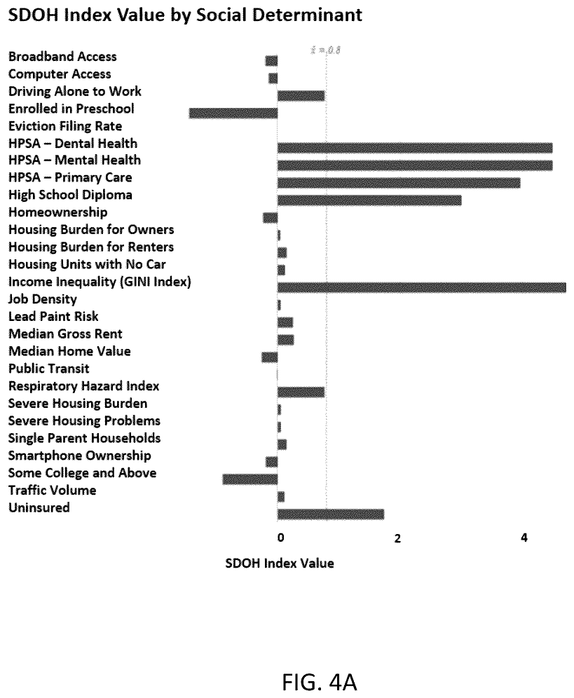

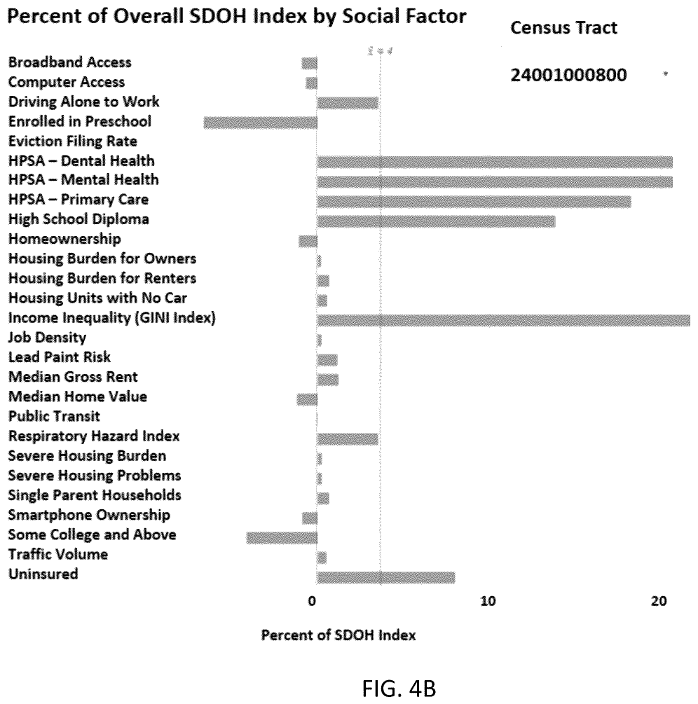

In one or more embodiments, FIG. 4 may indicate, based on the data obtained from data sources 210, 310, the effect of the SDOH or other factors on the health outcomes. For example, in an embodiment, a plurality of the SDOH or other factors may be identified and analyzed by the health risk analytics system 200, 300. The SDOH or other factors may be selected by the user or any number of SDOH or other factors may be included for analysis to determine which SDOH or other factors have the greatest impact on the health outcomes, both positively and negatively.

As seen in FIG. 4, the SDOH or other factors that have the greatest negative impact on the health outcomes were income inequality, lack of insurance, and health profession shortage areas. In an embodiment, the determination whether a SDOH or other factor effects health outcomes includes identifying the SDOH or other factors that have a deviation value greater than 0.8, but can also include using values over 0.5 or the like. The value for determining the effect of the SDOH is not intended to be limiting in scope, but rather provided as an example thereof.

FIG. 4 also shows that certain SDOH or other factors may have a positive influence on the health outcome. For example, in an embodiment, childhood enrolment in Pre-K may improve the overall life expectancy or improve the quality of life of the person. The SDOH or other factors having the highest SDOH Risk Index values may then be used to determine the vulnerability scores, as discussed above, e.g., by calculating the health vulnerability score by adding the SDOH or other factors having the highest impact and weighting the SDOH Risk Index values.

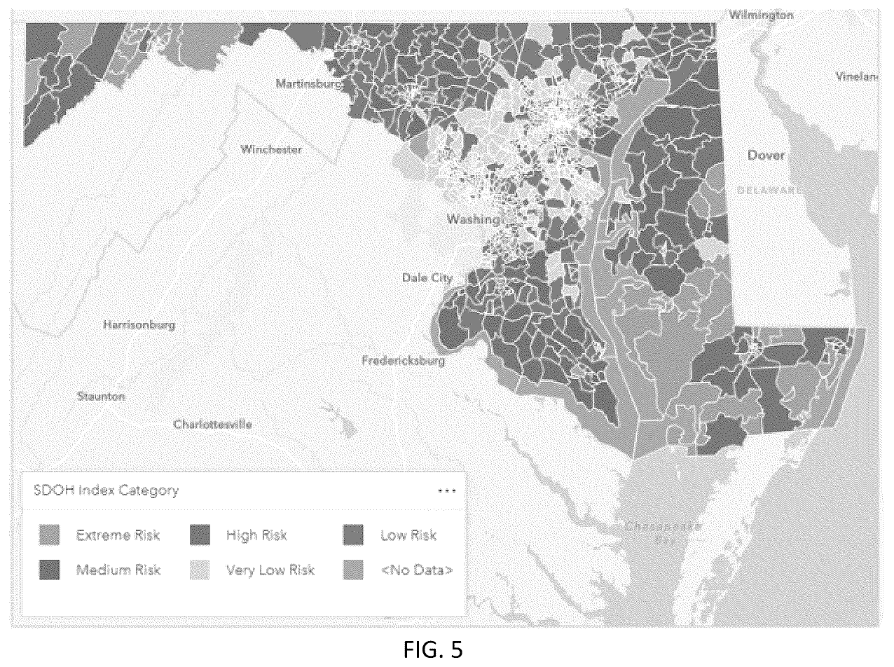

In one or more embodiments, FIG. 5 may indicate regions having high vulnerability scores based on the SDOH or other factors having the greatest impact on health outcomes. In an embodiment, the localities of interest having the greatest amount of SDOH or other factors that negatively affect health outcomes are identified by the shaded areas (showing the Health Risk Index). Such view provides decision makers actionable health risk insights to make determinations on how best to mitigate certain SDOH or other factors in the localities of interest having extreme and/or high risk.

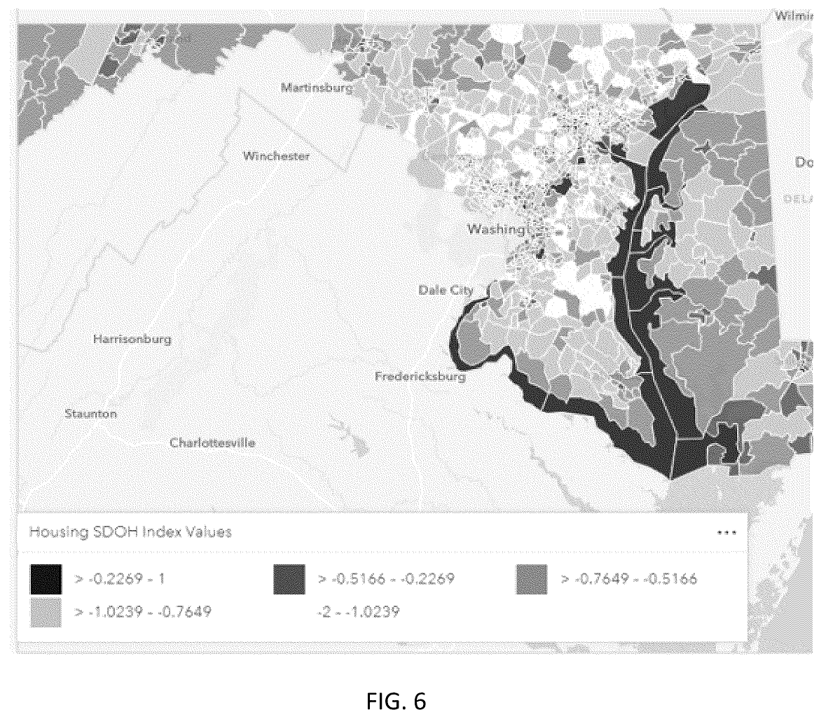

In a non-limiting embodiment, since the health risk analytics system may continuously stream data, the thematic map of FIG. 5 may automatically switch to the thematic map of FIG. 6, if a specific SDOH or other factor is found to most adversely affect a health outcome, e.g., a greater impact of the SDOH or other factor on the health vulnerability score. For example, in an embodiment, a change in housing risk factors, a flood, or the like may trigger the switch of the thematic map so that public health decision makers may take an appropriate action to mitigate the effect of the SDOH or other factor on the health outcome, e.g., divert resources to the areas having SDOH Index values of greater than −0.2269 to 1.

That is, in an embodiment, not only do FIGS. 5 and 6 illustrate thematic mapping of the health vulnerability scores for localities of interest, the figures also illustrate that the thematic mapping via GUIs may change in which the localities of interest having the highest health vulnerability scores are changed or updated and the associated new SDOH or other factors having the highest SODH Risk Index values may be displayed.

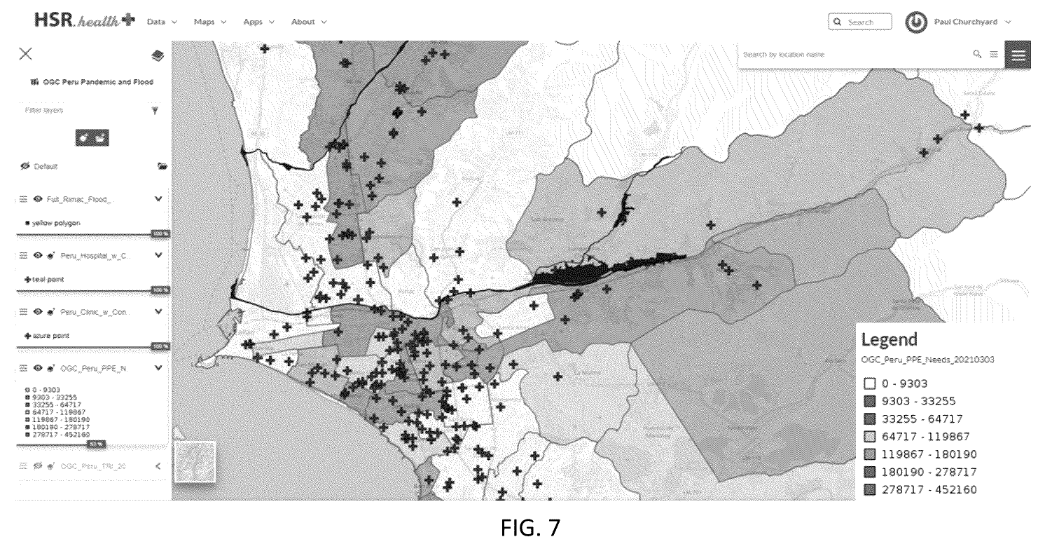

Similarly, in one or more embodiments, FIG. 7 may indicate regions having high health vulnerability scores based on the SDOH or other factors having the greatest impact on health outcomes. In an embodiment, the localities of interest having the greatest amount of SDOH or other factors that negatively affect health outcomes are identified by the shaded areas (showing the Health Risk Index). FIG. 7 also illustrates health facilities, in which pop-up images may be provided to provide information on each health facility. Such view provides decision makers actionable health risk insights to make determinations on how best to mitigate certain SDOH or other factors in the localities of interest having extreme and/or high risk, e.g., based on PPE needs for a particular region.

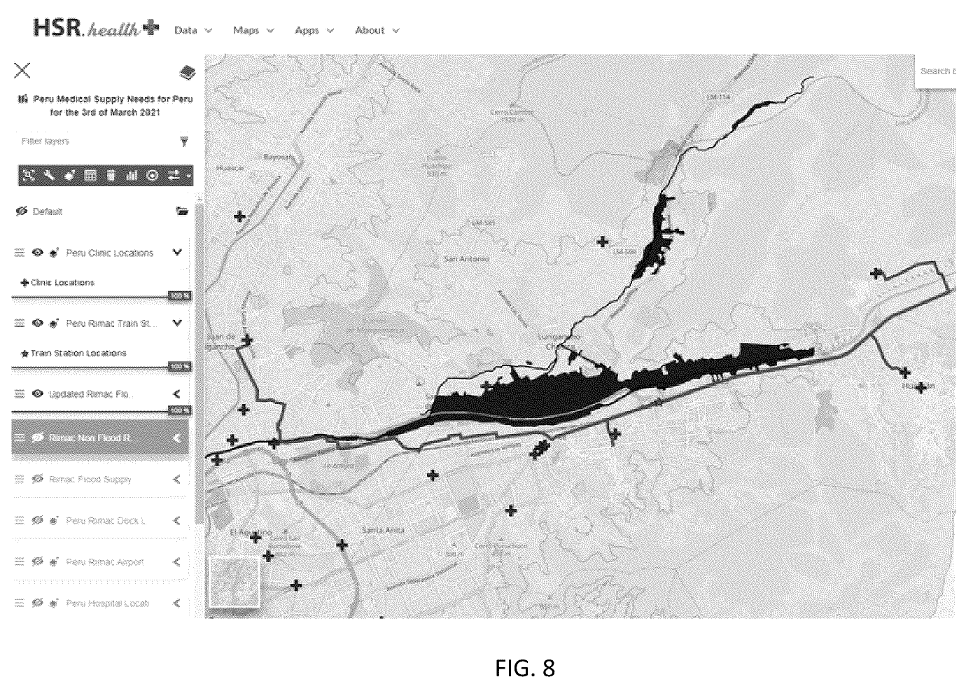

Similarly, in a non-limiting embodiment, since the health risk analytics system may continuously stream data, the thematic map of FIG. 7 may automatically switch to the thematic map of FIG. 8, if a specific SDOH or other factor is found to most adversely affect a health outcome, e.g., a greater impact of the SDOH or other factor on the health vulnerability score. For example, in an embodiment, a natural disaster, e.g., a landslide or flood, or the like may trigger the switch of the thematic map so that public health decision makers may take an appropriate action to mitigate the effect of the SDOH or other factor on the health outcome. For example, in an embodiment, FIG. 8 is automatically changed from FIG. 7, so that the decision makers may be focused on the specific locality of interest, e.g., river basin, and health facilities that may need additional medical supplies, so that the decision makers may take the appropriate mitigation actions at the granular level.

FIGS. 4-8, of course, illustrate but examples of how the tabling and mapping may provide a ready visualization of the health vulnerability score for localities of interest that are of greater risk. With knowledge of demographic, economic, and other social determinants, relationships between and among the subject population and social determinants are also readily ascertained by this visual presentation, much more effectively than mere data manipulation or mental analysis, which may not be effectively performed for the myriad and disparate data, data sources, and correlations that form the table and thematic mapping shown in FIGS. 4-8.

As discussed above, the methods and system for the health risk analytics includes at least the following:

Identify the problem and its specific driving factors.

Identify the magnitude of the problem, relative to health outcomes.

Leverage the decision tree to design an intervention specific to the identified problem and underlying social factors having the greatest impact on health outcomes.

Target the interventions to the specific problem.

As such, the determination of the SDOH Risk Index and health vulnerability score(s) provide an overall view into health risks from social and economic factors overall, as well as the ability to aggregate the impact of the factors into themes (e.g., housing, education, income, transit) as well as down to the impact of an individual factor, such as lead exposure, that provides actionable health risk insights to decision makers and policy-makers. The SDOH Risk Index and health vulnerability score(s), therefore, provides a holistic tool that includes in its process flow measures for the collaboration and synergy with organizations outside of public health to implement solutions. Taken overall, the SDOH Risk Index and health vulnerability score(s) form a decision tree providing decision support to public health officials in the design and targeting of on-the-ground social interventions to improve health outcomes.

### Use Cases

Depending on the specific underlying SDOH or other factor that causes the risk of adverse health outcomes, the decision makers (e.g., users of the SDOH Risk Index and health vulnerability score(s)) may take specific actions depending on the SDOH or other factors found to have the greatest impact on health outcomes.

In an embodiment, when housing quality is a driving factor, e.g., has one of the greatest impacts on adverse health outcomes, health departments may work with local elected officials and economic development/redevelopment authorities to seek legislative support for housing repair assistance funds.

In an embodiment, when lead paint is the driving factor, e.g., has one of the greatest impacts on adverse health outcomes, health departments and health systems may work with local community organizations to subsidize painting programs to address and/or mitigate the health risks from lead paint.

In another embodiment, when housing density is the driving factor, e.g., has one of the greatest impacts on adverse health outcomes, health systems may work with the local zoning and planning boards to build low-cost housing—including potentially tiny houses or container homes—in and around the specific neighborhoods where dense housing exists.

In yet another embodiment, when education attainment is the driving factor, e.g., has one of the greatest impacts on adverse health outcomes, health departments may work with public school systems and private schools to develop programs improving high school graduation rates as well as improving K-20 pathways, and/or work with public schools, after school programs, community outreach programs, and/or other outreach organizations to provide health literacy education and training programs.

In another embodiment, when transit is the driving factor, e.g., has one of the greatest impacts on adverse health outcomes, there are a number of solutions public health decision makers may pursue. For example, the decision maker may work with the transportation department in developing health-friendly public transit schedules, work with the transportation department to include health facility accessibility in their long-term road planning, collaborate with Uber/Lyft and other ride sharing solutions for non-emergency transportation, work with state and federal Medicaid programs, as well as insurers, to cover non-emergency transportation to and from hospitals and scheduled medical visits, or establish a non-emergency medical transportation voucher program. If long commute is the driving factor having an impact on adverse health outcomes, then public health systems may work with local communities, transportation departments, employers, and community service organizations to advocate for: work-from-home programs, hybrid work schedules, or traffic abatement programs such as HOV lanes, one-directional lanes to mitigate traffic to/from employment centers.

In an embodiment, policies around the HRSA-run program for payment of clinician student loans may include the SDOH Risk Index and/or health vulnerability score to better address areas that will benefit the most from additional clinical staff. As such, the SDOH Risk Index and health vulnerability score may be integrated into this program by being included into the data underlying the maps HRSA currently uses to identify locations that qualify for tuition relief. These maps are intended to show areas of insufficient clinician coverage, but do not take social factors into account such that decision makers may allocate healthcare benefits by geographic location with the intention of improving health equity and the SDOH Index can make those programs more effective.

In another embodiment, many states use a Certificate of Need process to control the distribution of healthcare facilities throughout their state with the intention to ensure the right level of care facilities are available to meet the anticipated health and medical need of the state population. While well-intentioned, the true level of health and medical need cannot be ascertained without complete knowledge of the social, economic, and environmental factors impacting health outcomes. Specifically, the SDOH Risk Index and/or health vulnerability score(s) may tell which populations and communities are at greater risk of readmissions or higher length of stay—all which greatly influence the level of healthcare facility need.

In still another embodiment, the health posture of a population and a nation is a key driver of its economic potential. Healthcare is also a key expense for governments and businesses, as well as for individuals, families and communities. As such, the SDOH Risk Index and/or health vulnerability score may be taken into account by decision makers for economic policy ranging from redevelopment efforts, transportation funding, zoning considerations, housing policy such that they are designed with health concerns in mind to ensure the policies contribute towards improving health conditions. For example, the SDOH Risk Index and/or health vulnerability scores may be integrated into economic policy development process by inclusion into the data underlying the development process for those programs, as well as by visual inspection of the SDOH Index Map. That is, social and economic factors are known to adversely impact health outcomes, in which there is overlap both in social factors impacting health outcomes as well as leading to the need for community policing (e.g., crime, loitering, public nuisances, etc.). Efforts to reduce those social and economic factors can both improve the quality of life within those communities, as well as improve health outcomes. In an embodiment, the SDOH Risk Index and/or health vulnerability score may be integrated into Community Policing decisions including areas/neighborhoods for policing, size of force, duration of policing initiatives, as well as specific issues which to address by inclusion into the data underlying the decision process, as well as by visual inspection of the SDOH Index Map.

In other embodiment, the SDOH Risk Index and/or health vulnerability score may be used to identify the degree to which improvement in the social, economic, and/or environmental factor may improve the associated health outcome. For example, in an embodiment, the SDOH Risk Index and/or health vulnerability score may be used to predict that a 10% reduction, for example, in a specific SDOH can be expected to have a 20% reduction in the overall adverse health outcome.

In yet another embodiment, the SDOH Risk Index and/or health vulnerability score may be integrated into the data analysis efforts used by insurers and payers to design these programs and efforts in one of two ways:

Determining the effect of the social, economic, and/or environmental factors on health outcomes, which impact economic conditions, and therefore economic development/redevelopment efforts should take into consideration the impact they may have in improving conditions leading to lowering healthcare costs. Such lowered costs can be considered in both selecting economic development/redevelopment efforts as well as in allocating funds to those efforts. They can be included in the ROI of such efforts as well.

The SDOH Risk Index and/or health vulnerability score may also be used to inform which and the degree of health costs will be improved by a specific development/redevelopment effort, it can also help select or rank potential efforts on their impact on health costs overall.

In an embodiment, the SDOH Risk Index and/or health vulnerability score may also be integrated into the data analysis efforts used for the development/redevelopment efforts. Visual inspection of the SDOH Index Map can also guide this process from global or national to very granular local levels.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments. Additionally, while the above has been discussed with respect to methods and systems, it is appreciated that the methods may be stored on non-transitory computer-readable medium having computer-readable instructions, which when executed by a processor, performs the above steps of operation.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

The herein described subject matter sometimes illustrates different components contained within, or connected with, different other components. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A method of providing health risk analytics, comprising:
obtaining a set of social, economic, and/or environmental data from at least one data source for localities of interest;
removing source identification information from the set of social, economic, and/or environmental data;
obtaining adverse health outcomes for the localities of interest;
identifying an impact of social, economic, and/or environmental factors on the adverse health outcomes based on the set of social, economic, and/or environmental data;
determining which of the social, economic, and/or environmental factors have a greatest impact on the adverse health outcomes for a group of people in the localities of interest to determine a social determinants of health risk index for the localities of interest;
determining a health vulnerability score for the group of people in the localities of interest for the adverse health outcomes based on the social determinants of health risk index; and
displaying an effect of the social, economic, and/or environmental factors having the greatest impact on the adverse health outcomes for the group of people in the localities of interest having highest health vulnerability scores.

2. The method of claim 1, further comprising:
removing any geospatial data from the set of social, economic, and/or environmental data.

3. The method of claim 1, wherein the obtaining the set of social, economic, and/or environmental data is through at least one of an application programming interface, webscraping, direct download from a source site, and cloud objects.

4. The method of claim 1, wherein the identifying the impact of social, economic, and/or environmental factors on the adverse health outcomes includes transforming the set of social, economic, and/or environmental data into a risk signal associated with the set of social, economic, and/or environmental data.

5. The method of claim 1, wherein the localities of interest include at least one of a nation, a state, a region, a county, a ZIP code, a Census tract, or a hospital service delivery area.

6. The method of claim 1, wherein the at least one data source includes a combination of one or more data sources selected from the following U.S. Census, U.S. Bureau of Labor and Statistics, U.S. Department of Transportation, OpenRx, the Center for Disease Control and Prevention, and Centers for Medicare and Medicaid Services.

7. The method of claim 1, wherein the social determinants of health comprise a combination of one or more of the following social determinants of health including childhood development, quality of life, education level, housing density, neighbourhood, family income, race, and ethnicity.

8. The method of claim 1, wherein the determining which of the social, economic, and/or environmental factors have the greatest impact includes leveraging a bi-variate cluster analysis to identify the specific social, economic, and/or environmental factors impacting the adverse health outcomes.

9. The method of claim 2, further comprising:
reattaching the geospatial data to the health vulnerability score; and
providing a mapping and/or visual geospatial representation of the health vulnerability score at different geographical levels for the localities of interest.

10. The method of claim 1, further comprising:
predicting a triggering event from the social, economic, and/or environmental factors for the adverse health outcomes based on changes of the social, economic, and/or environmental factors.

11. The method of claim 1, wherein the obtaining the set of social, economic, and/or environmental data further includes processing image data.

12. The method of claim 11, wherein the processing of the image data includes converting the set of social, economic, and/or environmental data into tabular form after removing any of the geospatial data from the image data.

13. The method of claim 1, wherein data from the set of social, economic, and/or environmental data from the at least one data source is geocoded.

14. The method of claim 1, wherein the adverse health outcomes include at least one of mortality or years of life lost from mortality.

15. The method of claim 14, wherein the social, economic, and/or environmental factors include at least one of housing quality, housing density, education attainment, commute times, and transportation.

16. The method of claim 1, further comprising:
tracking the mitigating the effect of the social and/or environmental factors in a short term or a long term.

17. The method of claim 1, further comprising:
displaying the localities of interest having the highest health vulnerability scores and the social determinants of health associated with the highest health vulnerability scores; and
when the localities of interest change based on any change to the localities of interest having the highest health vulnerability scores, displaying any updated localities of interest having a new highest health vulnerability score and new social, economic, and/or environmental factors associated with the new highest health vulnerability score.

18. A non-transitory computer-readable medium having computer-readable instructions that, if executed by a computing device, cause the computing device to perform operations comprising the method of claim 1.

19. A system for providing health risk analytics comprising:
a plurality of data sources having social, economic, and/or environmental data;
a cloud-based system configured to obtain a set of the social, economic, and/or environmental data from the plurality of data sources, wherein the cloud-based system includes machine-learning algorithms, which are configured to:
remove source identification information from the set of social, economic, and/or environmental data,
obtain adverse health outcomes from localities of interest,
identify an impact of social, economic, and/or environmental factors on the adverse health outcomes based on the set of social, economic, and/or environmental data,
determine which of the social, economic, and/or environmental factors have a greatest impact on the adverse health outcomes for a group of people in the localities of interest to determine a social determinants of health risk index for the localities of interest, and
determine a health vulnerability score for the group of people in the localities of interest for the adverse health outcomes based on the social determinants of health risk index; and
a relational cloud-based server configured to receive the health vulnerability score for the localities of interest and the associated social, economic, and/or environmental factors having the greatest impact on the adverse health outcomes for the group of people, wherein the relational cloud-based server is configured to transmit the health vulnerability score for the localities of interest and the associated social, economic, and/or environmental factors to application program interfaces for display by an end user.

20. The system of claim 19, wherein the cloud-based system is further configured to:
remove any geospatial data from the set of social, economic, and/or environmental data; and
reattach the geospatial data to the health vulnerability score before transmitting the health vulnerability score to the relational cloud-based server.
